(19) United States
(12) Patent Application Publication
(10) Pub. No.: US 2017/0279168 A1
(43) Pub. Date: Sep. 28, 2017

(54) GAS DETECTOR AND ELECTROCHEMICAL ELEMENT WITH GAS DETECTOR

(71) Applicant: TDK CORPORATION, Tokyo (JP)

(72) Inventors: Tomohiko KATO, Tokyo (JP); Takayuki MARUYAMA, Tokyo (JP)

(73) Assignee: TDK CORPORATION, Tokyo (JP)

(21) Appl. No.: 15/467,474

(22) Filed: Mar. 23, 2017

(30) Foreign Application Priority Data
Mar. 28, 2016 (JP) 2016-063487

(51) Int. Cl. H01M 10/48; H01G 11/20; G01N 21/78; H01M 10/0525; G01N 33/00

(52) U.S. Cl. CPC H01M 10/488; H01M 10/0525; G01N 33/0047; G01N 21/783; H01G 11/20; H01M 2220/30

(57) ABSTRACT

A gas detector wherein a porous coordination polymer represented by formula (1) is supported on a supporter, the supporting amount of the porous coordination polymer per area is 0.02 mg/cm² or more and 0.3 mg/cm² or less,

Feₓ(pz)[Ni_{1-y}M_{y}(CN)₄] (1)

(wherein, pz=pyrazine, 0.95≦5≦1.05 , M=Pd or pt, 0≦y≦0.15).

The present invention relates to a gas detector and an electrochemical element with the gas detector.

### BACKGROUND

With the decrease in size and increase in functionality of portable electronic devices in recent years, further miniaturization, weight reduction and higher capacity are expected for electrochemical elements.

Electrochemical elements can be made into various forms. A prismatic type, a pillared type and a pouch type or the like can be listed as the representative ones.

Among them, the pouch-type electrochemical element uses a pouch-type case made by sheets such as aluminum laminate film or the like, thus, it is light and can be manufactured into various forms. There is also a strong point in the simple manufacturing process. On the other hand, compared with the pillared type and the prismatic type, the pouch-type has a problem that it is easy to swell due to a flaw or an increase in the inner pressure.

In the electrochemical elements, in a lithium ion secondary battery or a lithium ion capacitor, a mixed solvent of a ring carbonate such as ethylene carbonate, a chain carbonate such as diethyl carbonate is usually used as the electrolyte solvent; in the electric, double layer capacitor, acetonitrile, propylene carbonate or the like is used as the electrolyte solvent; and in aluminum electrolytic capacitor, ethylene glycol or the like is used as the electrolyte solvent. When the sealability of the case of the electrochemical element is insufficient or when a pinhole or the like occurs in the case, a part of these solvents will become vapor and be evaporated and there will be problems such as had smells leaked from the hermetically sealed container or deterioration of the properties.

Various detection methods for leaked gas from the hermetically sealed container are proposed up to the present.

For example, in Patent document 1, a detection method is proposed for detecting the leaked detected gas from the hermetic battery using a gas sensor by manufacturing a hermetic battery in a hermetically sealed container with the detected gas atmosphere such as helium or argon or the like and then removing the detected gas in the hermetically sealed container followed by decompressing.

However, in the detection method of Patent document 1, a hermetically sealed container is required in the manufacturing process, thus, not only the instrument will be in a large scale, but also a detected gas supplying, pressure reducing devices and processes such as sensing for detected gas using a sensor are required. Therefore, there is a problem that the detection cannot he carried out simply. Further, there is a problem that the gas leakage before or after the detecting process cannot be detected.

A self-assembled regular complex with a high molecular weight from metal ions and organic ligands is called as a porous coordination polymer. A Hofmann type porous coordination polymer has a structure with a grown jungle gym type skeleton and has countless spaces in its inner part. Thus, it is well known for its adsorption for various molecules or the like. In Non-Patent Documents 1 to 3, a phenomenon called as spin crossover is described which is happened in a porous coordination polymer with specific structure, wherein the magnetic property varies between two states which are called as high-spin state and low-spin state by external factors such as heat, light, the adsorption of molecule or the like. Gas detection can be carried out using this phenomenon, but there is a problem that the sensitivity is not sufficient in the determination of gas with low concentration.

### Patent Documents

Patent Document 1: JP2009-26569A

### Non-Patent Documents

Non-Patent Document 1: Inorganic Chemistry, 2001, Vol 40, p. 3838-3839.
Non-Patent Document 2: Angewante Chemie International Edition, 2008, Vol 47, p. 6433-6437.
Non-Patent Document 3: Journal of the American Chemical Society, 2009, Vol 131, p. 10998-11009.

### SUMMARY

The present invention is made in view of the above problems and aims to provide a. gas detector and an electrochemical element with the gas detector which has a better visibility and a better sensitivity than a conventional one.

The inventors of the present invention do a lot of researches and find that the above aim can be reached by using a gas detector characterized in that a porous coordination polymer represented by formula (1) is supported on a supporter wherein the supporting amount of the porous coordination polymer per area is 0.02 cm/mg² or more and 0.3 mg/cm² or less. And thereby the present invention is completed.

Feₓ(pz)[Ni_{1-y}M_{y}(CN)₄] (1) (pr=pyrazine)

(0.95≦x≦1.05, M=Pd or Pt, 0≦y'0.15)

That is, according to the present invention, the following inventions can be provided.

[1] A gas detector characterized in that a porous coordination polymer represented by formula (1) is supported on a supporter wherein the supporting amount of the porous coordination polymer per area is 0.02 mg/cm² or more and 0.3 mg/cm² or less.

Feₓ(pz)[Ni_{1-y}M_{y}(CN)₄] (1) (pr=pyrazine)

(0.95≦x≦1.05, M=Pd or Pt, 0≦y'0.15)

[2] The gas detector according to [1] characterized in that two or more regions are formed on the supporter with different supporting amounts of the porous coordination polymer per area

[3] An electrochemical element characterized in comprising the gas detector according to [1] or [2] in the vicinity of the surface wherein the electrochemical element uses an electrolyte containing volatile organic compounds.

According to the present invention, a gas detector and an electrochemical element with the gas detector having an excellent visibility and sensitivity can be provided.

### BRIEF DESCRIPTION OF THE DRAWINGS

FIG. 1 is a schematic view showing the primary chemical structure of the porous coordination polymer of the present invention.

FIG. 2 is a schematic view showing the gas detector of the present invention.

### DETAILED DESCRIPTION OF EMBODIMENTS

Hereinafter, the embodiments for carrying out the present invention will be described in detail with reference to the drawings. However, the present invention is not restricted by the contents disclosed in the following embodiments.

In the gas detector of the present embodiment, a porous coordination polymer represented by formula (1) is supported on a supporter wherein the supporting amount of the porous coordination polymer per area is 0.02 mg/cm² or more and 0.3 mg/cm² or less.

Feₓ(pz)[Ni_{1-y}M_{y}(CN)₄] (1) (pr=pyrazine)

(0.95≦x≦1.05, M=Pd or Pt, 0≦y'0.15)

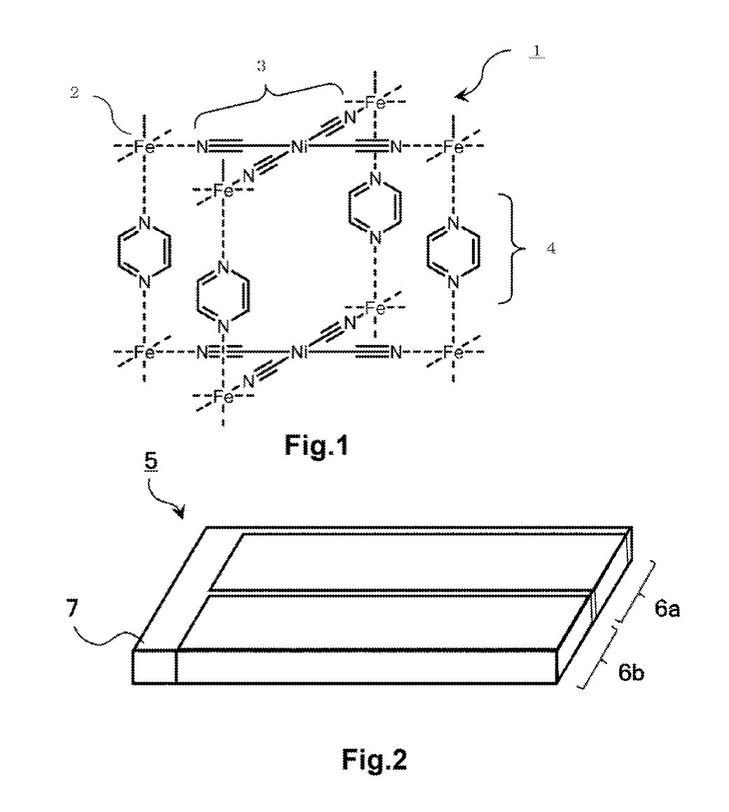

As shown in FIG. 1, porous coordination polymer 1 has a structure in which tetracyanonickelate ion 3 and pyrazine 4 are Self-assembly regularly coordinated onto ferrous ion 2 and the jungle gym type skeleton grown and the inner space can absorb varies of molecules or the like. In addition, a part of nickel can be replaced by at least one selected from palladium and platinum.

In porous coordination polymer 1, a phenomenon called as spin crossover is noticed, wherein the electron configuration of ferrous ions varies between two states which are called as high-spin state and low-spin state by external stimulations such as heat, pressure, or the adsorption of molecule. The spin variation can be considered to be in several tens of nano-seconds and has a character of a very high response speed.

The high-spin state refers to the state where the electrons are configured in a way that the spin angular momentum becomes the biggest according to the Hund's rule in the 5 orbits of the d electron of the ferrous ions in the complex. The low-spin state refers to the state where the electrons are configured in a way that the spin angular momentum becomes the smallest. The two states are different in the states of the electron and the crystal lattices, thus, the colors and the magnetisms of the complexes in the two states are different. That is, the gas can be detected with excellent visibility and sensitivity by using the spin crossover phenomenon caused by the adsorption of the molecule to the porous coordination polymer.

The porous coordination polymer in the high-spin state is orange and it will turn to reddish purple of the low-spin state if it is cooled sufficiently by liquid nitrogen or the like. In addition, if it is exposed in the gas of specific organic compounds such as acetonitrile or acrylonitrile or the like, the gas will be adsorbed into the inner of the crystal and turn to be the low-spin state. If the porous coordination polymer of reddish purple in the low-spin state is exposed in the organic compound gas which induces the high-spin state, it will take gas into the inner of the jungle gym type skeleton and turn to be orange of the high-spin state by the spin crossover phenomenon. As the gases of the organic compounds, vapors such as organic combustible gas or volatile organic solvent or the like can be listed as examples. That is, the porous coordination polymer in the low-spin state adsorbs gas(es) such as dimethyl carbonate (hereinafter, referred as DMC), diethyl carbonate (hereinafter, referred as DEC), and ethyl methyl carbonate (hereinafter, referred as EMC) or the like which is/are solvent(s) contained in the electrolyte for lithium ion secondary battery or lithium ion capacitor; or gas(es) such as ethylene and propylene or the like which is/are produced by the decomposition of the solvent(s) mentioned above; or further gas(es) such as propylene carbonate or the like which is solvent contained in the electric double layer capacitor; or further gas(es) such as ethylene glycol or the like which is solvent contained in the electrolyte for aluminum electrolytic capacitor, and turns to be orange in the high-spin state.

Refer to the composition of the porous coordination polymer of present embodiment, it can be confirmed by methods such as ICP (Inductively Coupled Plasma) Atomic Emission Spectroscopy, X-ray fluorescence elemental analysis, carbon/sulfur analysis and Oxygen/Nitrogen/Hydrogen Analysis or the like.

The spin state of the porous coordination polymer of the present embodiment can be confirmed by observing the response of the magnetization relative to the magnetic field using superconducting quantum interference device (SQUID) or vibrating sample magnetometer (VSM).

In the manufacture method of the porous coordination polymer of the present embodiment, first, carry out a reaction of the ferrous salt, antioxidant, tetracyanonickelate, tetracyanopalladate and tetracyanopiatinate in a proper solvent to obtain art intermediate. Then disperse the intermediate in a proper solvent and a precipitate can be precipitated by adding pyrazine into the dispersion liquid. The porous coordination polymer can be obtained by filtrating and drying the precipitate.

As the ferrous salt, ferrous sulfate heptahydrate, ammonium iron(II) sulfate hexahydrate or the like can be used. As the antioxidant, L-ascorbic acid or the like can be used. As the tetracyanonickelate, potassium tetracyanonickelate(II) hydrate or the like can be used. As the tetracyanopalladate, potassium tetracyartopalladate(II) hydrate or the like can be used. As the tetracyanoplatinate, potassium tetracyanoplatinate(II) hydrate or the like an be used.

As the solvent, methanol, ethanol, propanol, water and the like, or a mixed solvent thereof and the like can be used.

FIG. 2 is a schematic view showing the gas detector of the present embodiment. In FIG. 2, gas detector 5 is composed of porous coordination polymer 6a and porous coordination polymer 6b and supporter 7. The porous coordination polymer supporting amounts per area of porous coordination polymer 6a and porous coordination polymer 6b are different respectively.

If the supporting amount per area of the porous coordination polymers is 0.02 mg/cm² or more, the color change will be obvious when the detection gas is adsorbed onto the porous coordination polymer; if it is 0.3 mg/cm² or less, the color change will be obvious even if the detection gas is little and the visibility is excellent. When the supporting amount is 0.01 mg/cm² or less, a trend of unobviousness of the color change can be observed when, the detection gas is adsorbed by the porous coordination polymer. It is considered to be due to that it can be influenced easily by the influence of the color of the supporter or the influence of the atmospheric moisture or the volatile organic compounds. Further, when the supporting amount is 0.4 mg/cm² or more, a trend of unobviousness of the color change can be observed when the detection gas is less. It is considered to be due to the mottled presence of color-changed porous coordination polymer and porous coordination polymer which has not changed color. As described above, when the porous coordination polymer is supported on the supporter and the supporting amount of the porous coordination polymer per area is 0.02 mg/cm² or more and 0.3 mg/cm² or less, it can be used as a gas detector excellent in visibility and sensitivity.

In the gas detector of the, present embodiment, it is preferable that two or more regions with different supporting amounts of the porous coordination polymer per area are formed on the supporter. The two or more regions with different supporting amounts of the porous coordination polymer per area can be formed on the same surface of the supporter or on both of the front and back surfaces of the supporter. By forming two or more regions with different supporting amounts of the porous coordination polymer per area, the color tone of the region with a small supporting amount changes first and the visibility is improved comparing, with the color of the region whose color doesn't change where the supporting amount is large. Further, there may be a region in which the porous coordination polymer is not supported on the supporter.

Supporter 7 is not particularly restricted. For example, a cellulose-based cardboard such as a filter paper or the like or a paper filter can be used. In addition, the color of the supporter is preferred to be a color which is the complementary color relative to the color after the change caused by the adsorption of the detection gas by the porous coordination polymer, or white, grey or black because these colors can improve the visibility of the color change. Further, the thickness of the supporter is not particularly limited, but it is preferably 50 to 1000 μm from the viewpoint of easy handling during the manufacturing or using of the gas detector.

The supporting method of the porous coordination polymer onto the supporter is not particularly limited, a filtration method, a spray coating method, a brush coating method and a dip coating method can be listed.

(Measuring of the Supporting Amount of the Gas Detector)

The calculating method of the supporting amount of the porous coordination polymer per area of the gas detector of the present embodiment is, as follows.

The thin film fundamental parameter method of X-ray fluorescence analysis method is used to measure 10 points in the region where the porous coordination polymer of the detector is supported. The supporting amount of the porous coordination polymer is calculated from the obtained average supporting amount of Fe element. The measuring is performed using an instrument of ZSX 100e made by Rigaku Corporation, with a measuring spot diameter φ of 3 mm (SUS mask holder with a φ of 5 mm). The blank measurement value of the supporter is removed in the standard with difference intensity to determine the supporting amount of Fe element per area. The supporting amount of the porous coordination polymer is calculated from the amount ratio of the porous coordination polymer relative to the Fe element which is determined by the composition analysis of the porous coordination polymer.

The electrochemical element of the present embodiment characterized in that it uses an electrolyte containing a volatile organic compound and the gas detector is provided in the vicinity of the surface.

By providing the gas detector of the present embodiment in the vicinity of the surface of an electrochemical element using an electrolyte containing a volatile organic compound, when the sealability of the case of the electrochemical element is insufficient or when a pinhole or the, like occurs in the case, the leaked gas from the electrochemical element can be easily and sensitively detected by evaluating the color tone change of the gas detector.

By using the gas detector of the present embodiment, it is possible to detect the leaked gas from the electrochemical element even during processes other than the inspection process, or during transportation and storage, or the like.

### EXAMPLES

Hereinafter, the present invention is further specifically described based on the examples. However the present invention is restricted by the following examples.

### Example 1

(Preparation of the Porous Coordination Polymer)

Into an Erlenmeyer flask added with 240 mL of mixed solvent of distilled water and ethanol, 0.24 g of ammonium iron(II) sulfate hexahydrate, 0.1 g of L-ascorbic acid and 0.15 g of potassium tetracyanonickelate (ii) monohydrate were added and stirred. The precipitated intermediate particles were collected and 0.1 g of the obtained intermediate particles were dispersed in the ethanol and 0.10 g of pyrazine was added in to it using 30 minutes. The deposited precipitate was filtrated and dried, in atmosphere under 120° C. for 3 hours to obtain the orange porous coordination polymer.

(Manufacturing of the Gas Detector)

The porous coordination polymer of Example 1 was impregnated in acetonitrile under 25° C. for 10 hours. After that, it was suction-filtrated using filter paper No. 5C. and dried to form a reddish purple porous coordination polymer on the filter paper No. 5C. For the obtained reddish purple porous coordination polymer, the spin state was confirmed using superconducting quantum interference device (SQUID) and the result was low-spin state. Five milligram of the obtained porous coordination polymer was dispersed in 20 ml of acetonitrile to prepare a dispersion solution and the dispersion solution was placed in a spray bottle. Spray-coating was performed for three times on the filter paper No. 5C and then perform a vacuum drying at 25° C. to complete the gas detector.

(Measurement for the Supporting Amount of the Gas Detector)

As for the supporting amount of the porous coordination polymer per area of the obtained gas detector, it was measured by the X-ray fluorescence analysis method mentioned above and the result was 0.1 mg/cm².

(Detection of Diethyl Carbonate Gas)

A small fan and the gas detector were put into a Tedlar bag of 5 L. Nitrogen containing DEC was fed into it to obtain a concentration of 5 ppm. The gas detector was confirmed to turn orange after 72 minutes. On the other hand, in the case where only nitrogen was fed, a color tone change could note confirmed. Thereby, it could be confirmed that. DEC can be detected by evaluating the change of the color tone of the gas detector.

### (Detection of Other Gases)

Replacing DEC, ethylene, propylene, toluene, xylene, acetone, ethyl acetate, tetrahydrofuran, methanol, ethanol, n-propanol, isopropanol, ethylene glycol, ammonia, dimethylamine, trimethylamine triethylamine, acetic acid, formaldehyde, acetaldehyde, diethyl ether, dimethyl carbonate (DMC) and ethyl methyl carbonate (EMC) and propylene carbonate was used, the change of the color tone of the gas detector was confirmed in the same way, and the result was that the color changed to be orange.

(Detection of the Leaked Gas of the Lithium Ion Secondary Battery)

Two lithium ion secondary batteries were prepared with a gas detector attached on the surface of the case of the lithium ion secondary batteries. Among these batteries, a pinhole was punched artificially on one of the batteries using needle to simulate the condition when a pinhole was existed on the case. The batteries were put into the Tedlar bags respectively and, sealed, and then placed for 72 minutes. The color tone change of the gas sensor of the lithium ion secondary battery formed with pinhole was evaluated and it was confirmed the color changed to be orange. 10 μL of gas in the Tedlar bag was fetched using a gas-tight syringe and the components were analyzed using a gas chromatograph, and as the result, about 5 ppm of DEC was detected. On the other hand, the gas in the Tedlar bags with the lithium ion secondary battery of which the gas detector did not change was fetched and the content was analyzed. As the result, no gas content from electrolyte solution could be detected.

(Detection of the Leaked Gas of the Electric Double Layer Capacitor)

Two electric double layer capacitors were prepared with a gas detector attached on the surface of the case of the electric double layer capacitors. Among these capacitors, a pinhole was punched artificially on one of the capacitors using needle to simulate the condition when a pinhole was existed on the case. The capacitors were put into the Tedlar bags respectively and sealed, and then placed for 78 minutes. The color tone change of the gas sensor of the electric double layer capacitor formed with pinhole was evaluated and it was confirmed the color changed to be orange. 10 μL of gas in the Tedlar bag was fetched using a gas-tight syringe and the components were analyzed using a gas chromatograph, and as the result, about 8 ppm of propylene carbonate was detected. On the other hand, the gas in the Tedlar bags with the electric double layer capacitor of which the gas detector did not change was fetched and the content was analyzed. As the result, no gas content from electrolyte solution could be detected.

### Examples 2 to 4 and Comparative Example 1

Gas detectors were prepared in the same way as in Example 1 except that the number of spraying times was changed. The supporting amount of the porous coordination polymer calculated by the same way as in Example 1 and the time when the color tone change of the gas detector was visually observed were shown in Table 1.

### Comparative Example 2

A gas detector was prepared in the same way as in Example 1 except that the concentration of the acetonitrile dispersion solution of the porous coordination polymer and the number of spraying times was changed. The supporting amount of the porous coordination polymer calculated by the same way as in Example 1 and the time when the color tone change of the gas detector in the detection test of diethyl carbonate was visually observed were shown in Table 1.

TABLE 1

| | | | | Visually observed |
|---|---|---|---|---|
| | | Concentration | Supporting | time when the |
| | Composition of the porous | of DEC | amount | color tone change |
| | coordination polymer | (ppm) | (mg/cm²) | (minutes) |
| Example 1 | Fe_{0.99}(pz)[Ni_{1.0}(CN)₄] | 5 | 0.1 | 72 |
| Example 2 | Fe_{0.99}(pz)[Ni_{1.0}(CN)₄] | 5 | 0.3 | 82 |
| Example 3 | Fe_{0.99}(pz)[Ni_{1.0}(CN)₄] | 5 | 0.02 | 65 |
| Example 4 | Fe_{0.99}(pz)[Ni_{1.0}(CN)₄] | 5 | 0.2 | 75 |
| Comparative | Fe_{0.99}(pz)[Ni_{1.0}(CN)₄] | 5 | 0.4 | Unobviousness, |
| example 1 | | | | could not be |
| | | | | visually observed |
| Comparative | Fe_{0.99}(pz)[Ni_{1.0}(CN)₄] | 5 | 0.01 | Unobviousness, |
| example 2 | | | | could not be |
| | | | | visually observed |

(pz = pyrazine)

(Detection of Diethyl Carbonate Gas)

For the gas detectors of Examples 2 to 4, color tone change caused by diethyl carbonate gas was evaluated in the same way as Example 1, and as the result, it was confirmed that the gas detection portions turned to be orange. For the gas detectors of Comparative examples 1 and 2, color tone change caused by diethyl carbonate gas was evaluated in the same way as Example 1, and as the result, the color tone changes after 100 minutes were unobviousness and thus could not be visually observed.

### Example 5

(Manufacturing of the Gas Detector)

Acetonitrile dispersion solution of the porous coordination polymer prepared in the same way as Example 1 was placed in a spray bottle. Spray-coating was performed for three times on one half region of a filter paper No. 5C and then perform a vacuum drying at 25° C. After that, spray-coating was performed for nine times on the region not been, coated and then perform a vacuum drying at 25° C. again to complete the gas detector. The supporting amount of the porous coordination polymer per area was calculated by the same way as Example 1. The result was that the supporting amount was 0.1 mg/cm² in the region where 3 times of spray-coating were performed and it was 0.3 mg/cm² in the region where 9 times of spray-coating were performed.

(Detection of Diethyl Carbonate Gas)

In the same way as Example 1, a small fan and the gas detector prepared m Example 5 were put into a Tedlar bag of 5 L. Nitrogen containing DEC was fed into it to obtain a concentration of 5 ppm After 72 minutes, the region where 3 times of spray-coating were performed could be confirmed to turn orange more easily than the region where 9 times of spray-coating were performed.

### Examples 6 to 15, Comparative examples 3 to 5

Porous coordination polymers and gas detectors were prepared in the same way as in Example 1 except that ammonium iron(II) sulfate hexahydrate, potassium tetracyanonickelate (II) monohydrate, potassium tetracyanopalladate(II) hydrate and potassium tetracyanoplatinate(II) hydrate were weighed to obtain the compositions as shown in Table 2. The supporting amount of the porous coordination polymer calculated by the same way as in Example 1 and the time when the color tone change of the gas detector was visually observed were shown in Table 2.

TABLE 2

| | | | | Visually observed |
|---|---|---|---|---|
| | | Concentration | Supporting | time when the color |
| | Composition of the porous | of DEC | amount | tone change |
| | coordination polymer | (ppm) | (mg/cm²) | (minutes) |
| Example 6 | Fe_{0.98}(pz)[Ni_{0.98}Pd_{0.02}(CN)₄] | 5 | 0.1 | 72 |
| Example 7 | Fe_{0.95}(pz)[Ni_{0.98}Pd_{0.02}(CN)₄] | 5 | 0.1 | 72 |
| Example 8 | Fe_{1.05}(pz)[Ni_{0.98}Pd_{0.02}(CN)₄] | 5 | 0.1 | 72 |
| Example 9 | Fe_{0.98}(pz)[Ni_{0.98}Pd_{0.02}(CN)₄] | 5 | 0.1 | 72 |
| Example | Fe_{0.98}(pz)[Ni_{0.98}Pt_{0.02}(CN)₄] | 5 | 0.1 | 72 |
| 10 | | | | |
| Example | Fe_{1.02}(pz)[Ni_{0.98}Pt_{0.02}(CN)₄] | 5 | 0.1 | 73 |
| 11 | | | | |
| Example | Fe_{0.98}(pz)[Ni_{0.94}Pt_{0.06}(CN)₄] | 5 | 0.1 | 74 |
| 12 | | | | |
| Example | Fe_{0.98}(pz)[Ni_{0.98}Pd_{0.01}Pt_{0.01}(CN)₄] | 5 | 0.1 | 73 |
| 13 | | | | |
| Example | Fe_{0.98}(pz)[Ni_{0.86}Pt_{0.09}(CN)₄] | 5 | 0.1 | 74 |
| 14 | | | | |
| Example | Fe_{0.98}(pz)[Ni_{0.86}Pt_{0.14}(CN)₄] | 5 | 0.1 | 74 |
| 15 | | | | |
| Comparative | Fe_{0.94}(pz)[Ni_{0.98}Pd_{0.02}(CN)₄] | 5 | 0.1 | Unobviousness, |
| example 3 | | | | could not be |
| | | | | visually observed |
| Comparative | Fe_{1.06}(pz)[Ni_{0.98}Pd_{0.02}(CN)₄] | 5 | 0.1 | Unobviousness, |
| example 4 | | | | could not be |
| | | | | visually observed |
| Comparative | Fe_{0.98}(pz)[Ni_{0.84}Pt_{0.16}(CN)₄] | 5 | 0.1 | Unobviousness, |
| example 5 | | | | could not be |
| | | | | visually observed |

(pz = pyrazine)

(Detection of Diethyl Carbonate Gas)

For the gas detectors of Examples 6 to 15, color tone change caused by diethyl carbonate gas was evaluated in the same way as Example 1, and as the result, it was confirmed that the gas detection portions turned to be orange. For the gas detectors of Comparative examples 3 to 5, color tone change caused by diethyl carbonate gas was evaluated in the same way as Example 1, and as the result, the color tone changes after 100 minutes were unobviousness and thus could not be visually observed.

### Example 16

(Manufacturing of the Gas Detector)

A gas detector prepared in the same way as in Example 1 was heated wider 70° C. for one hour and thus a gas detector was manufactured in which the porous coordination polymer was turned to be orange under a high-spin state.

(Detection of the Leaked Gas of the Electric Double Layer Capacitor)

Two electric double layer capacitors were prepared with the gas detector of Example 16 attached on the surface of the case of the electric double layer capacitors containing acetonitrile in the electrolyte. Among these capacitors, a pinhole was punched artificially on one of the capacitors using needle to simulate the condition when a pinhole was existed on the case. The capacitors were put into the Tedlar bags respectively and sealed, and then placed for 3 minutes. The color tone change of the gas sensor of the electric double layer capacitor formed with pinhole was evaluated and it was confirmed the color changed to be reddish purple. 10 μL of gas in the Tedlar bag was fetched using a gas-tight syringe and the components were analyzed using a gas chromatograph, and as the result, about 20 ppm of acetonitrile was detected. On the other hand, the gas in the Tedlar bags with the electric double layer capacitor of which the gas detector did not change was fetched and the content was analyzed. As the result, no gas content from electrolyte solution could be detected.

It could be known from the results above that, the gas detectors of the examples are excellent in visibility and sensitivity. Further, gas can be detected easily with excellent sensitivity by using the electrochemical element provided with a gas detector of the examples.

### DESCRIPTION OF REFERENCE NUMERALS

1 . . . Porous coordnation polymer
2 . . . Ferrous ion

3 . . . Tetracyanonickelate ion

4 . . . Pyrazine

5 . . . detector

6a . . . Porous coordination polymer a

6b . . . Porous coordination polymer b

7 . . . Supporter

1. A gas detector wherein a porous coordination polymer represented by formula (1) is supported on a supporter,
the supporting amount of the porous coordination polymer per area is 0.02 mg/cm² or more and 0.3 mg/cm² or less,
Feₓ(pz)[Ni_{1-y}M_{y}(CN)₄] (1)
pr=pyrazine
0.95≦x≦1.05, M=Pd or Pt, 0≦y'0.15

2. The gas detector according to claim 1, wherein,
two or more regions are formed on the supporter with different supporting amounts of the porous coordination polymer per area.

3. An electrochemical element comprising the gas detector according to claim 1 in the vicinity of the surface, wherein the electrochemical element uses an electrolyte containing volatile organic compounds.

4. An electrochemical element comprising the gas detector according to claim 2 in the vicinity of the surface, wherein the electrochemical element uses an electrolyte containing volatile organic compounds
